(19) United States
(12) Patent Application Publication
(10) Pub. No.: US 2026/0283587 A1
(43) Pub. Date: Sep. 24, 2026

(54) ULTRASOUND DIAGNOSTIC APPARATUS AND METHOD OF CONTROLLING ULTRASOUND DIAGNOSTIC APPARATUS

(71) Applicant: FUJIFILM Corporation, Tokyo (JP)

(72) Inventor: Yu OBARA, Kanagawa (JP)

(73) Assignee: FUJIFILM Corporation, Tokyo (JP)

(21) Appl. No.: 19/568,747

(22) Filed: Mar. 16, 2026

(30) Foreign Application Priority Data
Mar. 24, 2025 (JP) 2025-048481

(51) Int. Cl. A61B 8/06; A61B 8/00

(52) U.S. Cl. CPC A61B 8/065; A61B 8/488; A61B 8/5223; A61B 8/58

(57) ABSTRACT

Provided are an ultrasound diagnostic apparatus and a method of controlling an ultrasound diagnostic apparatus that can easily obtain a highly reliable left ventricular outflow tract diameter.

An ultrasound diagnostic apparatus includes: a candidate value acquisition unit that acquires an outflow tract diameter candidate value in a left ventricular outflow tract based on a first ultrasound image in which a predetermined first cross section of a heart of a subject is captured; a blood flow measurement value acquisition unit that acquires a blood flow measurement value related to a blood flow rate based on a second ultrasound image in which a second cross section of the heart of the subject different from the first cross section is captured; a virtual cardiac output calculation unit that calculates a virtual cardiac output based on the outflow tract diameter candidate value and the blood flow measurement value; a reliability determination unit that determines reliability of the outflow tract diameter candidate value based on the virtual cardiac output; and a notification unit that notifies a user of the determined reliability.

### CROSS-REFERENCE TO RELATED APPLICATIONS

The present application claims priority under 35 U.S.C. § 119 to Japanese Patent Application No. 2025-048481, filed on Mar. 24, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a method of controlling an ultrasound diagnostic apparatus.

### 2. Description of the Related Art

In the related art, a so-called cardiac output is calculated by capturing an ultrasound image representing a tomographic plane of a heart of a subject by a so-called ultrasound diagnostic apparatus and analyzing the captured ultrasound image. The cardiac output is usually calculated by performing calculation steps of (1) measuring a left ventricular outflow tract diameter in a first ultrasound image representing a so-called parasternal left ventricular long axis view at a mid-systolic phase of the heart, and calculating a cross-sectional area of the left ventricular outflow tract, (2) calculating a blood flow velocity-time integral of the left ventricular outflow tract using a so-called pulse Doppler method by using a second ultrasound image representing a so-called apical five-chamber view or a so-called apical three-chamber view, (3) calculating a so-called stroke volume by multiplying the cross-sectional area of the left ventricular outflow tract by the blood flow velocity-time integral of the left ventricular outflow tract, and (4) calculating the cardiac output by multiplying the stroke volume by a heart rate. This technique is disclosed in, for example, JP1993-003869A (JP-H5-003869A).

### SUMMARY OF THE INVENTION

The left ventricular outflow tract diameter may not be accurately measured due to, for example, a position of an ultrasound probe that captures the first ultrasound image representing the parasternal left ventricular long axis view being deviated from an appropriate position, a phase of a frame used for measuring the left ventricular outflow tract diameter being deviated from the mid-systolic phase, or a boundary of the left ventricular outflow tract in the first ultrasound image being unclearly drawn due to some cause. Since the stroke volume and the cardiac output are proportional to a square of the left ventricular outflow tract diameter, it is required to measure the left ventricular outflow tract diameter with the accuracy of the order of millimeters. Therefore, in a case in which the left ventricular outflow tract diameter cannot be accurately measured, the stroke volume or the cardiac output may not be accurately measured, and thus it may be necessary to re-measure the left ventricular outflow tract diameter, which may make it difficult to quickly perform an examination.

The present invention has been made to solve such problems in the related art, and an object of the present invention is to provide an ultrasound diagnostic apparatus and a method of controlling an ultrasound diagnostic apparatus that can easily obtain a highly reliable left ventricular outflow tract diameter.

The above object can be achieved with the following configurations.

[1] An ultrasound diagnostic apparatus comprising: a candidate value acquisition unit that acquires an outflow tract diameter candidate value in a left ventricular outflow tract based on a first ultrasound image in which a predetermined first cross section of a heart of a subject is captured; a blood flow measurement value acquisition unit that acquires a blood flow measurement value related to a blood flow rate based on a second ultrasound image in which a second cross section of the heart of the subject different from the first cross section is captured; a virtual cardiac output calculation unit that calculates a virtual cardiac output based on the outflow tract diameter candidate value acquired by the candidate value acquisition unit and the blood flow measurement value acquired by the blood flow measurement value acquisition unit; a reliability determination unit that determines reliability of the outflow tract diameter candidate value acquired by the candidate value acquisition unit based on the virtual cardiac output calculated by the virtual cardiac output calculation unit; and a notification unit that notifies a user of the reliability determined by the reliability determination unit.

[2] The ultrasound diagnostic apparatus according to [1], in which the blood flow measurement value acquisition unit acquires a blood flow velocity-time integral as the blood flow measurement value.

[3] The ultrasound diagnostic apparatus according to [1] or [2], in which the virtual cardiac output calculation unit calculates a stroke volume or a cardiac output as the virtual cardiac output.

[4] The ultrasound diagnostic apparatus according to any one of [1] to [3], in which the reliability determination unit determines the reliability of the outflow tract diameter candidate value based on a comparison result between the virtual cardiac output calculated by the virtual cardiac output calculation unit and a range of a predetermined specified value.

[5] The ultrasound diagnostic apparatus according to any one of [1] to [3], in which the reliability determination unit determines normality of the blood flow measurement value by comparing the blood flow measurement value acquired by the blood flow measurement value acquisition unit with a predetermined measurement value, and determines the reliability of the outflow tract diameter candidate value based on both a determination result of the normality of the blood flow measurement value and a comparison result between the virtual cardiac output calculated by the virtual cardiac output calculation unit and a range of a predetermined specified value.

[6] The ultrasound diagnostic apparatus according to any one of [1] to [3], in which the reliability determination unit quantitatively determines the reliability of the outflow tract diameter candidate value based on a difference between the virtual cardiac output calculated by the virtual cardiac output calculation unit and a predetermined specified value.

[7] The ultrasound diagnostic apparatus according to any one of [1] to [3], in which the reliability determination unit determines the reliability of the outflow tract diameter candidate value based on a normalized cardiac output obtained by normalizing a difference between the virtual cardiac output and a predetermined specified value by the predetermined specified value, and a normalized blood flow measurement value obtained by normalizing a difference between the blood flow measurement value acquired by the blood flow measurement value acquisition unit and a predetermined measurement value by the predetermined measurement value.

[8] The ultrasound diagnostic apparatus according to [7], in which the reliability determination unit determines the reliability of the outflow tract diameter candidate value based on an absolute value of a difference between the normalized cardiac output and the normalized blood flow measurement value.

[9] The ultrasound diagnostic apparatus according to any one of [1] to [8], in which the candidate value acquisition unit acquires a plurality of the outflow tract diameter candidate values, the virtual cardiac output calculation unit calculates a plurality of the virtual cardiac outputs corresponding to the plurality of outflow tract diameter candidate values, and the reliability determination unit determines the reliability for each of the plurality of outflow tract diameter candidate values.

[10] The ultrasound diagnostic apparatus according to [9], further comprising: a monitor, in which the notification unit generates a graph showing a relationship between the plurality of outflow tract diameter candidate values and the plurality of virtual cardiac outputs, and displays the generated graph on the monitor.

[11] A method of controlling an ultrasound diagnostic apparatus, the method comprising: acquiring an outflow tract diameter candidate value in a left ventricular outflow tract based on a first ultrasound image in which a predetermined first cross section of a heart of a subject is captured; acquiring a blood flow measurement value related to a blood flow rate based on a second ultrasound image in which a second cross section of the heart of the subject different from the first cross section is captured; calculating a virtual cardiac output based on the outflow tract diameter candidate value and the blood flow measurement value; determining reliability of the outflow tract diameter candidate value based on the virtual cardiac output; and notifying a user of the reliability.

According to the aspects of the present invention, since the ultrasound diagnostic apparatus comprises: a candidate value acquisition unit that acquires an outflow tract diameter candidate value in a left ventricular outflow tract based on a first ultrasound image in which a predetermined first cross section of a heart of a subject is captured; a blood flow measurement value acquisition unit that acquires a blood flow measurement value related to a blood flow rate based on a second ultrasound image in which a second cross section of the heart of the subject different from the first cross section is captured; a virtual cardiac output calculation unit that calculates a virtual cardiac output based on the outflow tract diameter candidate value acquired by the candidate value acquisition unit and the blood flow measurement value acquired by the blood flow measurement value acquisition unit; a reliability determination unit that determines reliability of the outflow tract diameter candidate value acquired by the candidate value acquisition unit based on the virtual cardiac output calculated by the virtual cardiac output calculation unit; and a notification unit that notifies a user of the reliability determined by the reliability determination unit, so that a highly reliable left ventricular outflow tract diameter can be easily obtained.

### BRIEF DESCRIPTION OF THE DRAWINGS

FIG. 1 is a block diagram illustrating a configuration of an ultrasound diagnostic apparatus according to an embodiment of the present invention.

FIG. 2 is a block diagram illustrating an internal configuration of a transmission/reception circuit according to the embodiment of the present invention.

FIG. 3 is a block diagram illustrating an internal configuration of an image generation unit according to the embodiment of the present invention.

FIG. 4 is a diagram illustrating an example of a first ultrasound image representing a parasternal left ventricular long axis view.

FIG. 5 is a diagram illustrating an example of a second ultrasound image representing an apical five-chamber view.

FIG. 6 is a diagram illustrating an example of a plurality of outflow tract diameter candidates of a left ventricular outflow tract in the first ultrasound image.

FIG. 7 is a block diagram illustrating an internal configuration of a blood flow measurement value acquisition unit according to the embodiment of the present invention.

FIG. 8 is a diagram illustrating an example of a Doppler gate set in the second ultrasound image.

FIG. 9 is a diagram illustrating a graph showing a relationship between the outflow tract diameter candidate and a virtual cardiac output displayed on a monitor and an example of the first ultrasound image.

FIG. 10 is a flowchart illustrating an operation of the ultrasound diagnostic apparatus according to the embodiment of the present invention.

### DESCRIPTION OF THE PREFERRED EMBODIMENTS

Hereinafter, an embodiment of the invention will be described with reference to the accompanying drawings.

The following configuration requirements are described based on a representative embodiment of the present invention, but the present invention is not limited to such an embodiment.

In this specification, a numerical range represented by “to” means a range including numerical values described before and after “to”, both ends inclusive, as a lower limit value and an upper limit value.

In this specification, the terms “same” and “identical” include an error range generally allowed in the technical field.

### EMBODIMENT

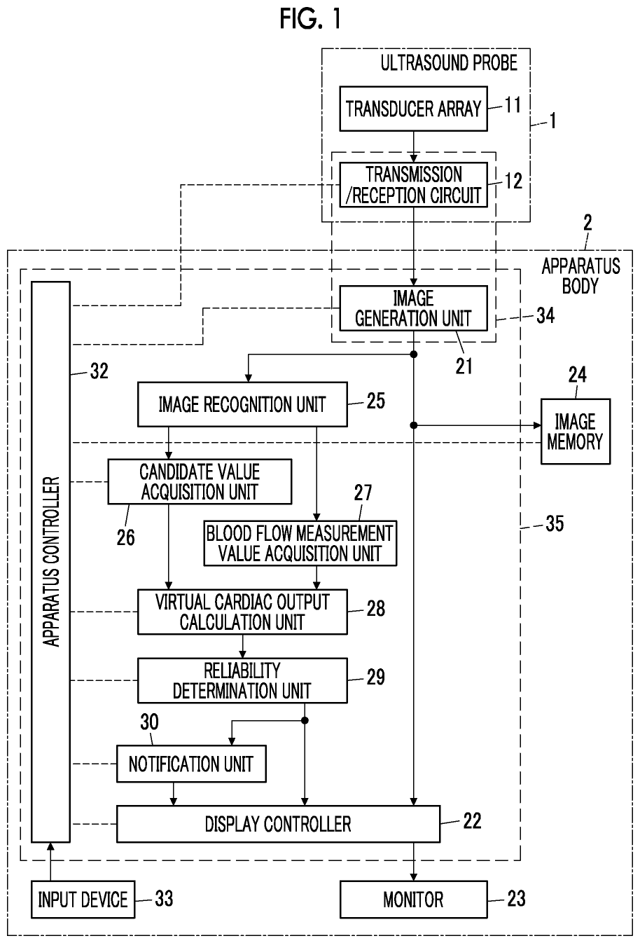

FIG. 1 illustrates a configuration of an ultrasound diagnostic apparatus according to the embodiment of the present invention. The ultrasound diagnostic apparatus comprises an ultrasound probe 1 and an apparatus body 2 that are connected to each other via so-called wired communication or so-called wireless communication.

The ultrasound probe 1 comprises a transducer array 11, and a transmission/reception circuit 12 that is connected to the transducer array 11.

The apparatus body 2 comprises an image generation unit 21 that is connected to the transmission/reception circuit 12. In the apparatus body 2, a display controller 22 and a monitor 23 are sequentially connected to the image generation unit 21. Further, an image memory 24 and an image recognition unit 25 are connected to the image generation unit 21. A candidate value acquisition unit 26 and a blood flow measurement value acquisition unit 27 are connected to the image recognition unit 25. A virtual cardiac output calculation unit 28 is connected to the candidate value acquisition unit 26 and the blood flow measurement value acquisition unit 27. A reliability determination unit 29 is connected to the virtual cardiac output calculation unit 28. A notification unit 30 is connected to the reliability determination unit 29. The reliability determination unit 29 and the notification unit 30 are each connected to the display controller 22. In addition, an apparatus controller 32 is connected to the transmission/reception circuit 12, the image generation unit 21, the display controller 22, the image memory 24, the image recognition unit 25, the candidate value acquisition unit 26, the blood flow measurement value acquisition unit 27, the virtual cardiac output calculation unit 28, the reliability determination unit 29, and the notification unit 30. An input device 33 is connected to the apparatus controller 32.

The transmission/reception circuit 12 and the image generation unit 21 form an image acquisition unit 34. In addition, a processor 35 for the apparatus body 2 is configured by the image generation unit 21, the display controller 22, the image recognition unit 25, the candidate value acquisition unit 26, the blood flow measurement value acquisition unit 27, the virtual cardiac output calculation unit 28, the reliability determination unit 29, the notification unit 30, and the apparatus controller 32.

The transducer array 11 of the ultrasound probe 1 includes a plurality of ultrasound oscillators arranged in a one-dimensional or a two-dimensional manner. In accordance with a drive signal supplied from the transmission/reception circuit 12, each of the ultrasound oscillators transmits an ultrasonic wave, receives an ultrasound echo from the subject, and outputs a signal based on the ultrasound echo. Each of the ultrasound oscillators is configured by forming, for example, electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 34, which is composed of the transmission/reception circuit 12 and the image generation unit 21, acquires a plurality of frames of ultrasound images as a moving image in which a heart of the subject is captured, by transmitting and receiving the ultrasound beams using the ultrasound probe 1.

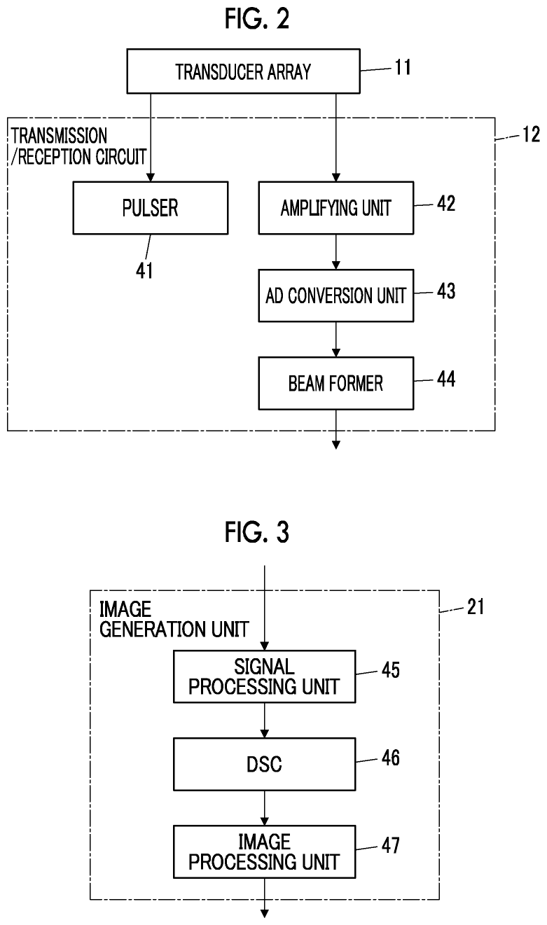

The transmission/reception circuit 12 transmits the ultrasonic waves from the transducer array 11 and generates a beamformed signal based on reception signals acquired by the transducer array 11 under control of the apparatus controller 32. As illustrated in FIG. 2, the transmission/reception circuit 12 includes a pulser 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, and the pulser 41 adjusts an amount of delay of each drive signal such that the ultrasound transmitted from the plurality of ultrasound oscillators of the transducer array 11 form the ultrasound beams, based on a transmission delay pattern selected in accordance with a control signal from the apparatus controller 32, and supplies the adjusted signal to the plurality of ultrasound oscillators. In a case in which a pulsed or continuous wave-like voltage is applied to the electrodes of the ultrasound oscillator of the transducer array 11, the piezoelectric material expands and contracts to generate pulsed or continuous wave-like ultrasound from each of the ultrasound oscillators, whereby the ultrasound beams are formed from the combined wave of the ultrasound.

The transmitted ultrasound beam is reflected by the target such as a part of the subject, and propagates toward the transducer array 11 of the ultrasound probe 1. The ultrasound echo propagating toward the transducer array 11 as described above is received by each of the ultrasound oscillators constituting the transducer array 11. In this case, each of the ultrasound oscillators constituting the transducer array 11 receives the propagating ultrasound echo to expand and contract to generate the reception signal, which is an electrical signal, and outputs these reception signals to the amplifying unit 42.

The amplifying unit 42 amplifies the signal input from each of the ultrasound oscillators constituting the transducer array 11, and transmits the amplified signal to the AD conversion unit 43. The AD conversion unit 43 converts the signal transmitted from the amplifying unit 42 into digital reception data. The beam former 44 performs so-called receive focus processing by applying respective delays to the reception data received from the AD conversion unit 43, and then adding the delayed data together. By this receive focus processing, each reception data, which is converted by the AD conversion unit 43, is added in phase, and the beamformed signal in which the focus of the ultrasound echo is narrowed down is acquired.

As illustrated in FIG. 3, the image generation unit 21 has a configuration in which a signal processing unit 45, a digital scan converter (DSC) 46, and an image processing unit 47 are sequentially connected in series.

The signal processing unit 45 generates a B-mode image signal, which is tomographic image information related to tissues inside the subject, by performing, on the beamformed signal received from the transmission/reception circuit 12, correction of the attenuation due to a distance in accordance with a depth of a reflection position of the ultrasound by using a sound velocity value set by the apparatus controller 32 and then performing envelope detection processing.

The DSC 46 converts (raster-converts) the B-mode image signal generated by the signal processing unit 45 into the image signal in accordance with a standard television signal scanning method.

The image processing unit 47 performs various necessary image processing, such as gradation processing, on the B-mode image signal input from the DSC 46, and then transmits the B-mode image signal to the display controller 22, the image memory 24, and the image recognition unit 25. Hereinafter, the B-mode image signal that has been image-processed by the image processing unit 47 will be referred to as an ultrasound image.

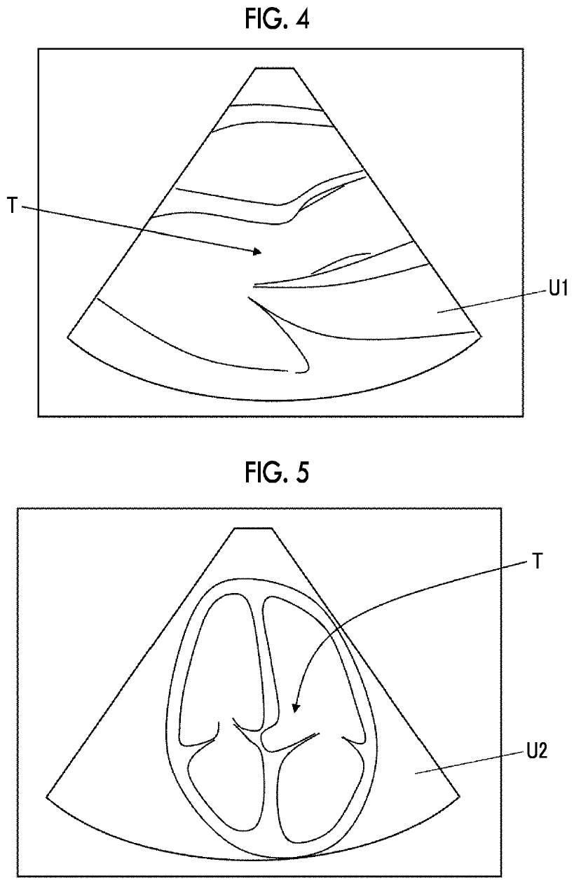

In the present invention, the image acquisition unit 34 acquires the plurality of frames of ultrasound images in which a first cross section and a second cross section of the heart of the subject, which are different from each other, are captured, respectively. For example, as schematically illustrated in FIG. 4, the image acquisition unit 34 can acquire an ultrasound image representing a so-called parasternal left ventricular long axis view as a first ultrasound image U1 representing the first cross section. The parasternal left ventricular long axis view is a cross section including a left ventricle, a left ventricular outflow tract T, a left atrium, and a right ventricle. In addition, for example, as schematically illustrated in FIG. 5, the image acquisition unit 34 can acquire an ultrasound image representing a so-called apical five-chamber view as a second ultrasound image U2 representing the second cross section. The apical five-chamber view is a cross section of the heart including five lumens of the left ventricle, the left ventricular outflow tract T, the left atrium, the right ventricle, and a right atrium. Although not illustrated, the image acquisition unit 34 can also acquire an ultrasound image representing a so-called apical three-chamber view instead of the second ultrasound image U2 representing the apical five-chamber view. The apical three-chamber view is a cross section of the heart including three lumens of the left ventricle, the left atrium, and the right ventricle. Both the apical five-chamber view and the apical three-chamber view include the left ventricular outflow tract T.

The image memory 24 is a memory in which the plurality of frames of ultrasound images acquired by the image acquisition unit 34 are stored. The user can use, for example, the plurality of frames of ultrasound images stored in the image memory 24 to confirm the ultrasound images after the examination. Here, for example, a recording medium such as a flash memory, a hard disk drive (HDD), a solid-state drive (SSD), a flexible disk (FD), a magneto-optical disk (MO disk), a magnetic tape (MT), a random-access memory (RAM), a compact disc (CD), a digital versatile disc (DVD), a secure digital card (SD card), or a universal serial bus memory (USB memory) can be used as the image memory 24.

The image recognition unit 25 recognizes whether the plurality of frames of ultrasound images acquired by the image acquisition unit 34 are ultrasound images in which the first cross section or the second cross section is captured. The image recognition unit 25 can recognize the ultrasound image by, for example, storing template data representing a typical image pattern of the parasternal left ventricular long axis view, the apical five-chamber view, and the apical three-chamber view in advance, and recognizing any of the parasternal left ventricular long axis view, the apical five-chamber view, or the apical three-chamber view by using a so-called template matching method of searching for the ultrasound image using the template data. The image recognition unit 25 can have, for example, a trained model in so-called machine learning, which has been trained using a large number of ultrasound images representing the parasternal left ventricular long axis view, the apical five-chamber view, and the apical three-chamber view, and can cause the trained model to output a recognition result by inputting the ultrasound image to the trained model. The image recognition unit 25 can also use algorithms such as scale-invariant feature transform (SIFT) and support vector machine (SVM).

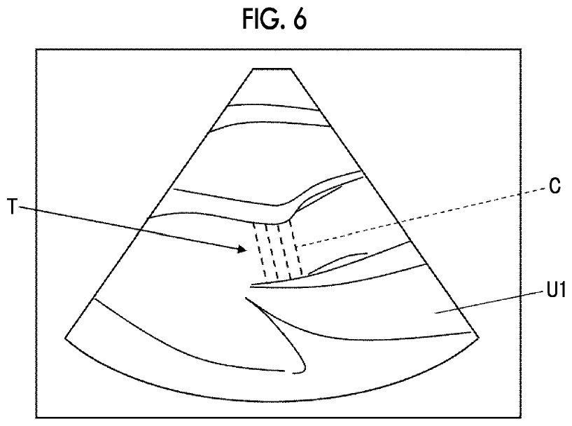

The candidate value acquisition unit 26 acquires an outflow tract diameter candidate value in the left ventricular outflow tract T based on the first ultrasound image U1 in which the first cross section of the heart of the subject is captured. The candidate value acquisition unit 26 can detect the aortic valve connected to the left ventricular outflow tract T by analyzing the first ultrasound image U1, and acquire outflow tract diameter candidate values C at a plurality of positions within a predetermined distance, for example, within 1 cm from the detected aortic valve toward the left ventricle, as illustrated in FIG. 6. In this case, the candidate value acquisition unit 26 can detect the left ventricular outflow tract T and the aortic valve by, for example, a brightness difference between the inside and the outside of the left ventricular outflow tract T. In addition, the candidate value acquisition unit 26 can also detect the left ventricular outflow tract T and the aortic valve by, for example, a method using the trained model in machine learning. In addition, the candidate value acquisition unit 26 can also acquire a plurality of outflow tract diameter candidate values C based on a position of an anatomical structure other than the aortic valve, such as a so-called mitral valve and the left ventricle, present around the left ventricular outflow tract T.

The candidate value acquisition unit 26 can calculate a running direction of the left ventricular outflow tract T using, for example, a so-called thinning-out algorithm, and acquire the plurality of outflow tract diameter candidate values in a direction orthogonal to the calculated running direction.

The candidate value acquisition unit 26 can dispose a plurality of positions in the left ventricular outflow tract T for acquiring the plurality of outflow tract diameter candidate values at predetermined equal intervals. Further, the candidate value acquisition unit 26 can determine the plurality of positions in the left ventricular outflow tract T for acquiring the plurality of outflow tract diameter candidate values such that the plurality of outflow tract diameter candidate values become different from each other.

The candidate value acquisition unit 26 can acquire the plurality of outflow tract diameter candidate values at the plurality of positions set by the user via the input device 33.

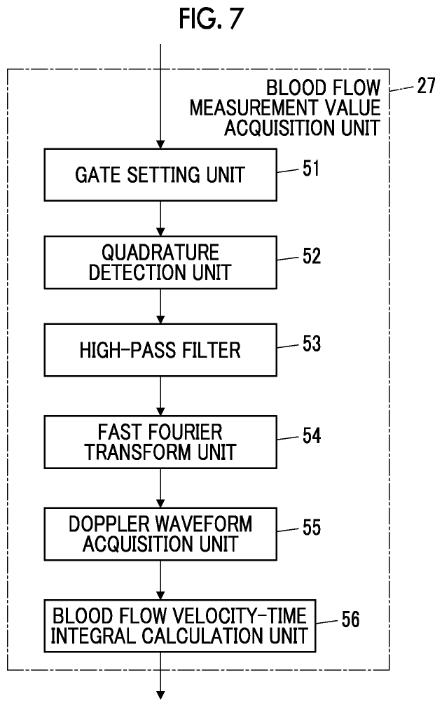

The blood flow measurement value acquisition unit 27 acquires a blood flow measurement value related to a blood flow rate based on the second ultrasound image U2 in which the second cross section of the heart of the subject different from the first cross section is captured. For example, the blood flow measurement value acquisition unit 27 can acquire, as the blood flow measurement value, a blood flow velocity-time integral. As illustrated in FIG. 7, the blood flow measurement value acquisition unit 27 can have a configuration in which a gate setting unit 51, a quadrature detection unit 52, a high-pass filter 53, a fast Fourier transform unit 54, a Doppler waveform acquisition unit 55, and a blood flow velocity-time integral calculation unit 56 are connected in series.

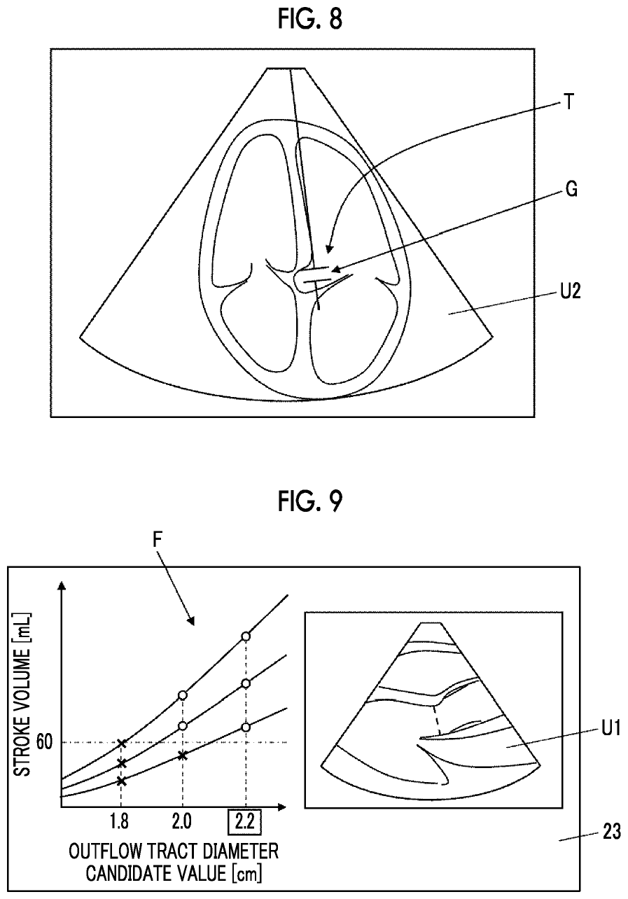

The gate setting unit 51 sets a so-called Doppler gate G in the left ventricular outflow tract T, for example, as illustrated in FIG. 8. The Doppler gate G is used for measuring the blood flow velocity in the Doppler gate G by a so-called pulse Doppler method. The gate setting unit 51 can automatically set the Doppler gate G in the left ventricular outflow tract T by detecting the left ventricular outflow tract T by, for example, a method of template matching or a method using the trained model in machine learning. Further, the gate setting unit 51 can also set the Doppler gate G at the position designated by the user via the input device 33.

The quadrature detection unit 52 mixes the beamformed signal received from the transmission/reception circuit 12 with the carrier signal of a reference frequency to perform quadrature detection on the beamformed signal and convert the beamformed signal into a complex signal.

The high-pass filter 53 functions as a so-called wall filter, and removes the frequency component resulting from the motion of the body tissue inside the subject, from the complex signal generated by the quadrature detection unit 52.

The fast Fourier transform unit 54 performs a Fourier transform on the complex signals of a plurality of sample points to perform frequency analysis, obtains the blood flow velocity, and generates a spectrum signal.

The Doppler waveform acquisition unit 55 acquires a Doppler waveform image signal by representing the magnitude of each frequency component with brightness while aligning the spectrum signals generated by the fast Fourier transform unit 54 on a time axis. The Doppler waveform image signal is called a so-called Doppler image, in which the time axis is shown on a horizontal axis, a Doppler shift frequency, that is, the flow velocity is shown on a vertical axis, and the brightness of the waveform represents the power in each frequency component.

The blood flow velocity-time integral calculation unit 56 calculates a velocity-time integral (VTI) based on the Doppler waveform acquired by the Doppler waveform acquisition unit 55. The blood flow velocity-time integral calculation unit 56 calculates the blood flow velocity-time integral by identifying a cardiac cycle period in the Doppler waveform, and time-integrating the value of the flow velocity in the Doppler waveform by the cardiac cycle period. The blood flow velocity-time integral calculation unit 56 can identify a waveform repetition unit in the Doppler waveform by analyzing the Doppler waveform, and identify a period to which the identified repetition unit belongs as the cardiac cycle period.

In addition, the blood flow velocity-time integral calculation unit 56 can calculate a so-called time averaged maximum (TAMAX) or a so-called time averaged mean (TAMEAN or TAM) of the left ventricular outflow tract blood flow as the blood flow measurement value. The time averaged maximum flow velocity is also called a time averaged maximum velocity (TAMV). The time averaged mean is also called a time averaged velocity (TAV).

The processing of calculating the blood flow measurement value by the quadrature detection unit 52, the high-pass filter 53, the fast Fourier transform unit 54, the Doppler waveform acquisition unit 55, and the blood flow velocity-time integral calculation unit 56 is performed after the transducer array 11 is driven by the drive signal to transmit the ultrasonic waves in a pulsed manner under the control of the transmission/reception circuit 12 and the apparatus controller 32.

The virtual cardiac output calculation unit 28 calculates a virtual cardiac output based on the outflow tract diameter candidate value C acquired by the candidate value acquisition unit 26 and the blood flow measurement value acquired by the blood flow measurement value acquisition unit 27. The virtual cardiac output calculation unit 28 can calculate a so-called stroke volume or cardiac output as the virtual cardiac output. The virtual cardiac output calculation unit 28 can calculate a stroke volume by, for example, assuming that a cross-sectional shape of the left ventricular outflow tract T perpendicular to a direction along the blood flow is circular, calculating a cross-sectional area of the left ventricular outflow tract T by the outflow tract diameter candidate value C, and multiplying the cross-sectional area of the left ventricular outflow tract T by the blood flow velocity-time integral. The virtual cardiac output calculation unit 28 can further calculate a cardiac output by multiplying the stroke volume by a heart rate. The virtual cardiac output calculation unit 28 can calculate, for example, a reciprocal of a period of the identified repeating waveform as the heart rate by identifying a repeating waveform in a time change waveform of the blood flow velocity acquired by the Doppler waveform acquisition unit 55.

The reliability determination unit 29 determines the reliability of the outflow tract diameter candidate value C acquired by the candidate value acquisition unit 26 based on the virtual cardiac output calculated by the virtual cardiac output calculation unit 28. The reliability determination unit 29 can determine the reliability of the outflow tract diameter candidate value C based on, for example, a comparison result between the virtual cardiac output calculated by the virtual cardiac output calculation unit 28 and a range of a predetermined specified value. The range of the specified value of the virtual cardiac output can be determined, for example, based on guidelines determined by the society, the facility such as the hospital, or statistical information. The reliability determination unit 29 can determine that the outflow tract diameter candidate value C is reliable in a case in which the virtual cardiac output is within the range of the specified value, and can determine that the outflow tract diameter candidate value C is not reliable in a case in which the virtual cardiac output is outside the range of the specified value.

The reliability determination unit 29 can determine normality of the blood flow measurement value by comparing the blood flow measurement value acquired by the blood flow measurement value acquisition unit 27 with a predetermined measurement value, and determine the reliability of the outflow tract diameter candidate value C based on both a determination result of the normality of the blood flow measurement value and a comparison result between the virtual cardiac output calculated by the virtual cardiac output calculation unit 28 and the range of the predetermined specified value. The predetermined measurement value of the blood flow measurement value can also be determined based on guidelines determined by the society, the facility such as the hospital, or statistical information, as in the range of the specified value. The reliability determination unit 29 can determine that the blood flow measurement value is normal in a case in which a difference between the predetermined measurement value and the blood flow measurement value is equal to or less than a difference threshold value, and determine that the blood flow measurement value is abnormal in a case in which the difference between the predetermined measurement value and the blood flow measurement value is larger than the difference threshold value. The reliability determination unit 29 can determine, for example, as shown in Table 1, that the outflow tract diameter candidate value C is reliable in a case in which the blood flow measurement value is normal and the virtual cardiac output is within the range of the specified value, and the outflow tract diameter candidate value C is not reliable in a case in which the blood flow measurement value is abnormal and the virtual cardiac output is outside the range of the specified value, as a result is consistent, and determine that the outflow tract diameter candidate value C is not reliable in a case other than the above-described case, as a result is inconsistent.

TABLE 1

| Blood flow | | |
|---|---|---|
| measurement | | Reliability of outflow tract |
| value | Virtual cardiac output | diameter candidate value |
| Normal | Within range of specified | ◯ |
| | value | |
| Abnormal | Outside range of specified | ◯ |
| | value | |
| Normal | Outside range of specified | X |
| | value | |
| Abnormal | Within range of specified | X |
| | value | |

The reliability determination unit 29 can quantitatively determine the reliability of the outflow tract diameter candidate value C based on a difference between the virtual cardiac output calculated by the virtual cardiac output calculation unit 28 and the predetermined specified value. The reliability determination unit 29 can determine that the outflow tract diameter candidate value C is reliable in a case in which the difference between the virtual cardiac output and the predetermined specified value is equal to or less than a predetermined difference threshold value, and determine that the outflow tract diameter candidate value C is not reliable in a case in which the difference between the virtual cardiac output and the predetermined specified value is larger than the predetermined difference threshold value.

In addition, the reliability determination unit 29 can determine the reliability of the outflow tract diameter candidate value based on a normalized cardiac output obtained by normalizing the difference between the virtual cardiac output and the predetermined specified value by the predetermined specified value, and a normalized blood flow measurement value obtained by normalizing the difference between the blood flow measurement value acquired by the blood flow measurement value acquisition unit 27 and the predetermined measurement value by the predetermined measurement value. In this case, the reliability determination unit 29 can determine the reliability of the outflow tract diameter candidate value C based on, for example, an absolute value of a difference between the normalized cardiac output and the normalized blood flow measurement value. Specifically, the reliability determination unit 29 can determine that the outflow tract diameter candidate value C is reliable in a case in which the absolute value of the difference between the normalized cardiac output and the normalized blood flow measurement value is equal to or less than a predetermined threshold value, and determine that the outflow tract diameter candidate value C is not reliable in a case in which the absolute value of the difference between the normalized cardiac output and the normalized blood flow measurement value is larger than the predetermined threshold value. The reliability determination unit 29 can calculate the normalized cardiac output by dividing the difference between the virtual cardiac output and the predetermined specified value by the predetermined specified value, and calculate the normalized blood flow measurement value by dividing the difference between the blood flow measurement value and the predetermined measurement value by the predetermined measurement value.

In addition, in a case in which the plurality of outflow tract diameter candidate values C are acquired by the candidate value acquisition unit 26 and the plurality of virtual cardiac outputs corresponding to the plurality of outflow tract diameter candidate values are calculated by the virtual cardiac output calculation unit 28, the reliability determination unit 29 can determine the reliability for each of the plurality of outflow tract diameter candidate values C.

In addition, in a case in which the plurality of outflow tract diameter candidate values C are acquired at each position in the left ventricular outflow tract T at a plurality of time phases within the cardiac cycle period by the candidate value acquisition unit 26 and the plurality of virtual cardiac outputs corresponding to the plurality of outflow tract diameter candidate values C at the plurality of time phases are calculated by the virtual cardiac output calculation unit 28, the reliability determination unit 29 can determine the reliability for each of the plurality of outflow tract diameter candidate values C, and determine the reliability of the outflow tract diameter candidate value C at each position in the left ventricular outflow tract T in accordance with the plurality of determination results.

In this case, the reliability determination unit 29 can finally determine the reliability of the outflow tract diameter candidate value C at each position by, for example, the plurality of determination results of the reliability for the plurality of outflow tract diameter candidate values C corresponding to the plurality of time phases in the cardiac cycle period. For example, in a case in which the determination result indicating that the outflow tract diameter candidate value C at a specific position in the left ventricular outflow tract T is reliable is larger than the determination result indicating that the outflow tract diameter candidate value C at the specific position is not reliable for the plurality of outflow tract diameter candidate values C corresponding to the plurality of time phases, the reliability determination unit 29 can make a final determination that the outflow tract diameter candidate value C at the specific position is reliable.

In addition, the reliability determination unit 29 can make, for example, a final determination that the outflow tract diameter candidate value C at a specific position in the left ventricular outflow tract T is reliable in a case in which all of the plurality of determination results of the reliability for the plurality of outflow tract diameter candidate values C corresponding to the plurality of time phases in the cardiac cycle period at the specific position in the left ventricular outflow tract T are the determination that the outflow tract diameter candidate value C is reliable, and make a final determination that the outflow tract diameter candidate value C at the specific position in the left ventricular outflow tract T is not reliable in a case in which there is a determination result indicating that the outflow tract diameter candidate value C is not reliable for one or more of the plurality of outflow tract diameter candidate values C.

In addition, the reliability determination unit 29 can determine the reliability of the outflow tract diameter candidate value C by using, for example, an average value of the plurality of outflow tract diameter candidate values C corresponding to the plurality of time phases in the cardiac cycle period instead of using the outflow tract diameter candidate value C corresponding to one time phase in the cardiac cycle period at the specific position in the left ventricular outflow tract T.

The notification unit 30 notifies the user of the reliability determined by the reliability determination unit 29. The notification unit 30 can display, for example, at least one of the first ultrasound image U1 used for determining the outflow tract diameter candidate value C, the graph showing the relationship between the outflow tract diameter candidate value C and the blood flow measurement value, or an indicator related to the reliability of the outflow tract diameter candidate value C on the monitor 23, in addition to the determination result of the reliability. The indicator related to the reliability of the outflow tract diameter candidate value C refers to, for example, an indicator such as the virtual cardiac output calculated to determine the reliability and the range of the specified value, and the difference between the virtual cardiac output and the specified value.

As a specific example, the notification unit 30 can display a graph F and the first ultrasound image U1 on the monitor 23, as illustrated in FIG. 9. The graph F shows three curves corresponding to the relationship between the outflow tract diameter candi date value C and the stroke volume at different heart rates. The outflow tract diameter candidate values C of 1.8 cm, 2.0 cm, and 2.2 cm on the horizontal axis of the graph F are measured at different positions. In addition, in the three curves of the graph F, the blood flow velocity-time integral is different for each heart rate. In this example, the range of the specified value for the stroke volume is set to a range of 60 mL or more, and the determination result indicates that the outflow tract diameter candidate value C of 2.2 cm at which the stroke volume is 60 mL or more in all of the three curves is reliable, and thus the character “2.2” that is the display corresponding to the outflow tract diameter candidate value C is emphasized by being surrounded by a frame.

Meanwhile, the left ventricular outflow tract diameter may not be accurately measured due to, for example, a position of an ultrasound probe 1 that captures the first ultrasound image U1 being deviated from an appropriate position, a phase of a frame used for measuring the left ventricular outflow tract diameter being deviated from the mid-systolic phase, or a boundary of the left ventricular outflow tract T in the first ultrasound image U1 being unclearly drawn due to some cause. Since the stroke volume and the cardiac output are proportional to the square of the left ventricular outflow tract diameter, it is required to measure the left ventricular outflow tract diameter with the accuracy of the order of millimeters. Therefore, in a case in which the left ventricular outflow tract diameter cannot be accurately measured, the stroke volume or the cardiac output may not be accurately measured, and thus it may be necessary to re-measure the left ventricular outflow tract diameter, which may make it difficult to quickly perform the examination.

The user can easily select the optimal outflow tract diameter candidate value C as the left ventricular outflow tract diameter having high reliability by confirming the notification by the notification unit 30. Therefore, the user can accurately and quickly calculate the stroke volume or the cardiac output by using the value of the selected outflow tract diameter candidate value C without requiring the re-measurement of the left ventricular outflow tract diameter.

The display controller 22 performs predetermined processing on the first ultrasound image U1 and the second ultrasound image U2 acquired by the image acquisition unit 34, the indicator related to the reliability of the outflow tract diameter candidate value C calculated by the reliability determination unit 29, and the notification content by the notification unit 30, under the control of the apparatus controller 32, and displays the results on the monitor 23.

The monitor 23 displays the first ultrasound image U1 and the second ultrasound image U2 acquired by the image acquisition unit 34, the indicator related to the reliability of the outflow tract diameter candidate value C calculated by the reliability determination unit 29, and the notification content by the notification unit 30, under the control of the display controller 22, and includes, for example, a display device such as a liquid crystal display (LCD) or an organic electroluminescence display (organic EL display).

The input device 33 is an input device used by the user to perform an input operation, and is configured by, for example, a device such as a keyboard, a mouse, a trackball, a touchpad, and a touch sensor disposed in a state of being superimposed on the monitor 23.

In this embodiment, each processing in the processor 35 is executed by any computer. Any computer may execute these kinds of processing by a processor as hardware, a program as software, or a combination of the processor and the program. In such a case, the processor is configured to execute various kinds of processing in this embodiment in cooperation with the program, and may function as each unit or each means in this embodiment. The execution order of the processing by the processor is not limited to the above-described order and may be changed as appropriate. Any computer may mean a general-purpose computer, a computer for specific use, a workstation, or another system that can execute each processing.

The processor 35 may be configured by one or more hardware components, and the type of hardware is not limited. For example, the processor 35 may be configured by a programmable logic device such as a central processing unit (CPU), a microprocessing unit (MPU), or a field programmable gate array (FPGA), a dedicated circuit for executing specific processing, such as an application-specific integrated circuit (ASIC), or hardware such as a graphics processing unit (GPU) or a neural processing unit (NPU). Further, the type of the hardware component may be a combination of different types of hardware components. In a case in which the plurality of hardware components are configured to execute one or a plurality of kinds of processing of a certain processor, the plurality of hardware components may be present in devices physically separated from each other or may be present in the same device. Further, in any of the embodiments, the order of each processing performed by the processor 35 is not limited to the order described above, and may be changed as appropriate. Hardware is implemented in a form of an electrical circuit (circuitry) in which circuit elements, such as semiconductor elements, are combined.

Furthermore, the program may be software such as firmware or microcode. The program may be, for example, a group of program modules, and each function may be implemented by a processor configured to execute each function. The program may be a program code or a plurality of code segments stored in one or more non-transitory computer-readable media (for example, a storage media and other storages). The program may be stored in the plurality of non-transitory computer-readable media existing in devices physically separated from each other. The program code or the code segment may represent a procedure, function, subprogram, routine, subroutine, module, software package, class, or any combination of instructions, data structures, or program statements. The program code or the code segment may be connected to another code segment or a hardware circuit by the transmission and reception of information, data, arguments, parameters, or contents in the memory.

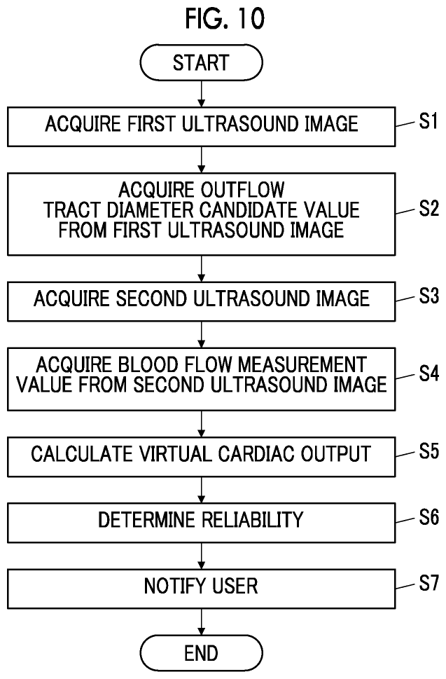

Hereinafter, an operation of the ultrasound diagnostic apparatus according to the embodiment will be described with reference to a flowchart illustrated in FIG. 10. Here, an example will be described in which the ultrasound image representing the parasternal left ventricular long axis view is acquired as the first ultrasound image U1 representing the first cross section, and then the ultrasound image representing the apical five-chamber view is acquired as the second ultrasound image U2 representing the second cross section.

In step S1, the user disposes the ultrasound probe 1 on the body surface of the subject to image the parasternal left ventricular long axis view, and the image acquisition unit 34 acquires the first ultrasound image U1 as illustrated in FIG. 4. In such a case, under the control of the apparatus controller 32, the transmission and reception of the ultrasound from the plurality of transducers of the transducer array 11 are started in accordance with the drive signal from the pulser 41 of the transmission/reception circuit 12 of the ultrasound probe 1, the ultrasound echo from the subject is received by the plurality of transducers of the transducer array 11, and the reception signal as the analog signal is output to the amplifying unit 42, is amplified, and then is subjected to the AD conversion via the AD conversion unit 43 to acquire the reception data.

The receive focus processing is performed on the reception data by the beam former 44, the beamformed signal generated by the receive focus processing is transmitted to the image generation unit 21 of the apparatus body 2, and thus the first ultrasound image U1 is generated by the image generation unit 21. In such a case, the signal processing unit 45 of the image generation unit 21 performs the correction of the attenuation in accordance with the depth of the reflection position of the ultrasound and the envelope detection processing on the beamformed signal, the DSC 46 performs the conversion into the image signal in accordance with the normal television signal scanning method, and the image processing unit 47 performs various types of necessary image processing such as gradation processing.

In step S2, the candidate value acquisition unit 26 acquires the outflow tract diameter candidate value in the left ventricular outflow tract T based on the first ultrasound image U1 acquired in step S1. The candidate value acquisition unit 26 can detect the aortic valve connected to the left ventricular outflow tract T by analyzing the first ultrasound image U1, and acquire outflow tract diameter candidate values C at a plurality of positions within a predetermined distance from the detected aortic valve toward the left ventricle, as illustrated in FIG. 6. In this case, the candidate value acquisition unit 26 can detect the left ventricular outflow tract T and the aortic valve by, for example, the brightness difference between the inside and the outside of the left ventricular outflow tract T, and also detect the aortic valve by a method using the trained model in machine learning.

In step S3, the user disposes the ultrasound probe 1 on the body surface of the subject to image the apical five-chamber view, and the image acquisition unit 34 acquires the second ultrasound image U2 as illustrated in FIG. 5 by the same processing as in step S1.

In step S4, the blood flow measurement value acquisition unit 27 acquires the blood flow measurement value related to the blood flow rate, based on the second ultrasound image U2 acquired in step S3. For example, the blood flow measurement value acquisition unit 27 can acquire, as the blood flow measurement value, the blood flow velocity-time integral. In this case, the blood flow measurement value acquisition unit 27 sets the Doppler gate G in the left ventricular outflow tract T, for example, as illustrated in FIG. 8, mixes the beamformed signal received from the transmission/reception circuit 12 with the carrier signal of the reference frequency to perform the quadrature detection on the beamformed signal and convert the beamformed signal into the complex signal, removes the frequency component derived from the movement of the tissue inside the body of the subject from the complex signal, performs frequency analysis by Fourier-transforming the complex signals of the plurality of sample points to obtain the blood flow velocity, and generates the spectrum signal. Further, the blood flow measurement value acquisition unit 27 acquires the Doppler waveform by representing the magnitude of each frequency component with brightness while aligning the spectrum signal on the time axis, and calculates the velocity-time integral based on the acquired Doppler waveform.

In step S5, the virtual cardiac output calculation unit 28 calculates the virtual cardiac output based on the outflow tract diameter candidate value C acquired in step S2 and the blood flow measurement value acquired in step S4. The virtual cardiac output calculation unit 28 can calculate the stroke volume or the cardiac output as the virtual cardiac output. The virtual cardiac output calculation unit 28 can calculate the stroke volume by, for example, assuming that the cross-sectional shape of the left ventricular outflow tract T perpendicular to the direction along the blood flow is circular, calculating the cross-sectional area of the left ventricular outflow tract T by the outflow tract diameter candidate value C, and multiplying the cross-sectional area of the left ventricular outflow tract T by the blood flow velocity-time integral. The virtual cardiac output calculation unit 28 can further calculate the cardiac output by multiplying the stroke volume by the heart rate. The virtual cardiac output calculation unit 28 can calculate, for example, the reciprocal of the period of the identified repeating waveform as the heart rate by identifying the repeating waveform in the time change waveform of the blood flow velocity acquired by the Doppler waveform acquisition unit 55.

In step S6, the reliability determination unit 29 determines the reliability of the outflow tract diameter candidate value C acquired in step S2 based on the virtual cardiac output calculated in step S5. The reliability determination unit 29 can determine that the outflow tract diameter candidate value C is reliable in a case in which the virtual cardiac output is within the range of the predetermined specified value, and determine that the outflow tract diameter candidate value C is not reliable in a case in which the virtual cardiac output is outside the range of the predetermined specified value. As described above, the reliability determination unit 29 can determine the reliability of the outflow tract diameter candidate value C under a certain criterion.

In step S7, the notification unit 30 notifies the user of the reliability determined in step S6. The notification unit 30 can display, for example, at least one of the first ultrasound image U1 used for determining the outflow tract diameter candidate value C, the graph showing the relationship between the outflow tract diameter candidate value C and the blood flow measurement value, or the indicator related to the reliability of the outflow tract diameter candidate value C on the monitor 23, in addition to the determination result of the reliability. The user can easily select the outflow tract diameter candidate value C that is reliable as the left ventricular outflow tract diameter used for the measurement by confirming the notification content of step S7, and can accurately calculate the stroke volume or the cardiac output by using the selected outflow tract diameter candidate value C.

In a case in which the processing of step S7 is completed in this way, the operation of the ultrasound diagnostic apparatus in accordance with the flowchart of FIG. 10 is completed.

As described above, with the ultrasound diagnostic apparatus according to the embodiment of the present invention, since the candidate value acquisition unit 26 acquires the outflow tract diameter candidate value C in the left ventricular outflow tract T based on the first ultrasound image U1 in which the first cross section of the heart of the subject is captured, the blood flow measurement value acquisition unit 27 acquires the blood flow measurement value related to the blood flow rate based on the second ultrasound image U2 in which the second cross section of the heart of the subject different from the first cross section is captured, the virtual cardiac output calculation unit 28 calculates the virtual cardiac output based on the outflow tract diameter candidate value C acquired by the candidate value acquisition unit 26 and the blood flow measurement value acquired by the blood flow measurement value acquisition unit 27, the reliability determination unit 29 determines the reliability of the outflow tract diameter candidate value C acquired by the candidate value acquisition unit 26 based on the virtual cardiac output calculated by the virtual cardiac output calculation unit 28, and the notification unit 30 notifies the user of the reliability determined by the reliability determination unit 29, the left ventricular outflow tract diameter having high reliability can be easily obtained.

In addition, the case has been described in which the transmission/reception circuit 12 is provided in the ultrasound probe 1, but the transmission/reception circuit 12 may be provided in the apparatus body 2.

Further, the case has been described in which the image generation unit 21 is provided in the apparatus body 2, but the image generation unit 21 may be provided in the ultrasound probe 1.

The apparatus body 2 may be a so-called stationary type, a portable type that is easily carried, or a so-called handheld type that is formed by, for example, a smartphone or a tablet type computer. As described above, the types of the devices forming the apparatus body 2 are not particularly limited.

Although the notification unit 30 notifies the user by the display on the monitor 23, the notification method by the notification unit 30 is not particularly limited to this. For example, in a case in which the ultrasound diagnostic apparatus comprises a speaker (not shown), the notification unit 30 can notify the user by a voice through the speaker.

In addition, although the example has been described in which the second ultrasound image U2 representing the apical five-chamber view is acquired after the first ultrasound image U1 representing the parasternal left ventricular long axis view is acquired, the first ultrasound image U1 may be acquired after the second ultrasound image U2 is acquired.

### EXPLANATION OF REFERENCES

1: ultrasound probe
2: apparatus body
11: transducer array
12: transmission/reception circuit
21: image generation unit
22: display controller
23: monitor
24: image memory
25: image recognition unit
26: candidate value acquisition unit
27: blood flow measurement value acquisition unit
28: virtual cardiac output calculation unit
29: reliability determination unit
30: notification unit
32: apparatus controller
33: input device
34: image acquisition unit
35: processor
41: pulser
42: amplifying unit
43: AD conversion unit
44: beam former
45: signal processing unit
46: DSC
47: image processing unit
51: gate setting unit
52: quadrature detection unit
53: high-pass filter
54: fast Fourier transform unit
55: Doppler waveform acquisition unit
56: blood flow velocity-time integral calculation unit
C: outflow tract diameter candidate value
F: graph
G: Doppler gate
T: left ventricular outflow tract
U1: first ultrasound image
U2: second ultrasound image

What is claimed is:

1. An ultrasound diagnostic apparatus comprising:
a processor configured to:
acquire an outflow tract diameter candidate value in a left ventricular outflow tract based on a first ultrasound image in which a predetermined first cross section of a heart of a subject is captured;
acquire a blood flow measurement value related to a blood flow rate based on a second ultrasound image in which a second cross section of the heart of the subject different from the first cross section is captured;
calculate a virtual cardiac output based on the outflow tract diameter candidate value and the blood flow measurement value;
determine reliability of the outflow tract diameter candidate value based on the virtual cardiac output; and
notify a user of the reliability.

2. The ultrasound diagnostic apparatus according to claim 1,
wherein the processor is configured to acquire a blood flow velocity-time integral as the blood flow measurement value.

3. The ultrasound diagnostic apparatus according to claim 1,
wherein the processor is configured to calculate a stroke volume or a cardiac output as the virtual cardiac output.

4. The ultrasound diagnostic apparatus according to claim 2,
wherein the processor is configured to calculate a stroke volume or a cardiac output as the virtual cardiac output.

5. The ultrasound diagnostic apparatus according to claim 1,
wherein the processor is configured to determine the reliability of the outflow tract diameter candidate value based on a comparison result between the virtual cardiac output and a range of a predetermined specified value.

6. The ultrasound diagnostic apparatus according to claim 2,
wherein the processor is configured to determine the reliability of the outflow tract diameter candidate value based on a comparison result between the virtual cardiac output and a range of a predetermined specified value.

7. The ultrasound diagnostic apparatus according to claim 3,
wherein the processor is configured to determine the reliability of the outflow tract diameter candidate value based on a comparison result between the virtual cardiac output and a range of a predetermined specified value.

8. The ultrasound diagnostic apparatus according to claim 1,
wherein the processor is configured to:
determine normality of the blood flow measurement value by comparing the blood flow measurement value with a predetermined measurement value, and
determine the reliability of the outflow tract diameter candidate value based on both a determination result of the normality of the blood flow measurement value and a comparison result between the virtual cardiac output and a range of a predetermined specified value.

9. The ultrasound diagnostic apparatus according to claim 2,
wherein the processor is configured to:
determine normality of the blood flow measurement value by comparing the blood flow measurement value with a predetermined measurement value, and
determine the reliability of the outflow tract diameter candidate value based on both a determination result of the normality of the blood flow measurement value and a comparison result between the virtual cardiac output and a range of a predetermined specified value.

10. The ultrasound diagnostic apparatus according to claim 3,
wherein the processor is configured to:
determine normality of the blood flow measurement value by comparing the blood flow measurement value with a predetermined measurement value, and
determine the reliability of the outflow tract diameter candidate value based on both a determination result of the normality of the blood flow measurement value and a comparison result between the virtual cardiac output and a range of a predetermined specified value.

11. The ultrasound diagnostic apparatus according to claim 1,
wherein the processor is configured to determine the reliability of the outflow tract diameter candidate value based on a difference between the virtual cardiac output and a predetermined specified value.

12. The ultrasound diagnostic apparatus according to claim 2,
wherein the processor is configured to determine the reliability of the outflow tract diameter candidate value based on a difference between the virtual cardiac output and a predetermined specified value.

13. The ultrasound diagnostic apparatus according to claim 3,
wherein the processor is configured to determine the reliability of the outflow tract diameter candidate value based on a difference between the virtual cardiac output and a predetermined specified value.

14. The ultrasound diagnostic apparatus according to claim 1,
wherein the processor is configured to determine the reliability of the outflow tract diameter candidate value based on a normalized cardiac output obtained by normalizing a difference between the virtual cardiac output and a predetermined specified value by the predetermined specified value, and a normalized blood flow measurement value obtained by normalizing a difference between the blood flow measurement value and a predetermined measurement value by the predetermined measurement value.

15. The ultrasound diagnostic apparatus according to claim 2,
wherein the processor is configured to determine the reliability of the outflow tract diameter candidate value based on a normalized cardiac output obtained by normalizing a difference between the virtual cardiac output and a predetermined specified value by the predetermined specified value, and a normalized blood flow measurement value obtained by normalizing a difference between the blood flow measurement value and a predetermined measurement value by the predetermined measurement value.

16. The ultrasound diagnostic apparatus according to claim 3,
wherein the processor is configured to determine the reliability of the outflow tract diameter candidate value based on a normalized cardiac output obtained by normalizing a difference between the virtual cardiac output and a predetermined specified value by the predetermined specified value, and a normalized blood flow measurement value obtained by normalizing a difference between the blood flow measurement value and a predetermined measurement value by the predetermined measurement value.

17. The ultrasound diagnostic apparatus according to claim 14,
wherein the processor is configured to determine the reliability of the outflow tract diameter candidate value based on an absolute value of a difference between the normalized cardiac output and the normalized blood flow measurement value.

18. The ultrasound diagnostic apparatus according to claim 1,
wherein the processor is configured to:
acquire a plurality of the outflow tract diameter candidate values;
calculate a plurality of the virtual cardiac outputs corresponding to the plurality of outflow tract diameter candidate values; and
determine the reliability for each of the plurality of outflow tract diameter candidate values.

19. The ultrasound diagnostic apparatus according to claim 18,
wherein the processor is configured to generate a graph showing a relationship between the plurality of outflow tract diameter candidate values and the plurality of virtual cardiac outputs; and
display the generated graph.

20. A method of controlling an ultrasound diagnostic apparatus, the method comprising:
acquiring an outflow tract diameter candidate value in a left ventricular outflow tract based on a first ultrasound image in which a predetermined first cross section of a heart of a subject is captured;
acquiring a blood flow measurement value related to a blood flow rate based on a second ultrasound image in which a second cross section of the heart of the subject different from the first cross section is captured;
calculating a virtual cardiac output based on the outflow tract diameter candidate value and the blood flow measurement value;
determining reliability of the outflow tract diameter candidate value based on the virtual cardiac output; and
notifying a user of the reliability.
